(19) United States
(12) Patent Application Publication
(10) Pub. No.: US 2024/0285339 A1
(43) Pub. Date: Aug. 29, 2024

(54) SENSORISED KNIFE-BLADE CUTTING SYSTEM

(71) Applicant: Norwegian University of Life Sciences, Ås (NO)

(72) Inventors: Alex MASON, Ås (NO); Olga KOROSTYNSKA, Ås (NO); Luis Eduardo CORDOVA-LOPEZ, Ås (NO)

(73) Assignee: Norwegian University of Life Sciences, Ås (NO)

(21) Appl. No.: 18/570,558

(22) PCT Filed: Jun. 10, 2022
(86) PCT No.: PCT/EP2022/065916

§ 371 Date: Dec. 14, 2023

(30) Foreign Application Priority Data
Jun. 17, 2021 (GB) 2108679.8

(51) Int. Cl. B26D 7/27; A22B 5/00; A61B 18/00; A61B 18/14; A61B 18/18

(52) U.S. Cl. CPC B26D 7/27; A22B 5/0041; A61B 18/1815; A61B 2018/00077; A61B 2018/00083; A61B 2018/0016; A61B 2018/00184; A61B 2018/00601; A61B 2018/00785; A61B 2018/00904; A61B 2018/1412; A61B 2018/1876

(57) ABSTRACT

A cutting system comprising a knife blade, a radio-frequency or microwave transmission system for transmitting a radio-frequency or microwave signal to the knife blade, a sensing system for sensing a parameter of a reflection of the radio-frequency or microwave signal and a processing system configured to analyse the parameter to determine a property of an environment adjacent the knife blade.

### BACKGROUND OF THE INVENTION

This invention relates to knife-blade cutting systems employing radio-frequency and microwave sensing systems and methods for operating the same.

Automation of processing steps is commonplace in the food industry to achieve high production efficiency. Although many food processing activities have successfully been automated, certain complex tasks, such as butchery, present particular challenges. The introduction of robots into such processing activities is difficult, as in general they lack “smart” tooling required to cope with advanced tasks such as those requiring as real-time adjustment of processes in response to changing conditions.

In the context of butchery, one approach for obtaining real-time feedback while an animal carcass is being cut is through the use of robot vision systems. However, it can be difficult for such systems always to maintain good visibility of a cutting tool and the carcass as it is being cut, which means that often operations are planned and then performed blind.

If more accurate sensing can be achieved, greater integration of robots into the butchery process will become possible, allowing significant improvements to be achieved, e.g. by allowing changing work patterns and new processes to be efficiently introduced. There is therefore a need to improve robot sensing systems for food processing applications.

The present invention seeks to provide a novel cutting system that can enable more accurate sensing.

### SUMMARY OF THE INVENTION

From a first aspect, the invention provides a cutting system comprising:
a knife blade;
a radio-frequency or microwave transmission system for transmitting a radio-frequency or microwave signal to the knife blade;
a sensing system for sensing a parameter of a reflection of the radio-frequency or microwave signal; and
a processing system configured to analyse the parameter to determine a property of an environment adjacent the knife blade.

From a second aspect, the invention provides a method of operating a knife blade, comprising:
transmitting a radio-frequency or microwave signal to a knife blade;
sensing a parameter of a reflection of the radio-frequency or microwave signal; and
analysing the parameter to determine a property of an environment adjacent the knife blade.

Thus it will be seen that, in accordance with the invention, a radio-frequency (RF) or microwave signal is transmitted through the knife blade of the cutting system, and couples with the environment adjacent to the knife blade so as to create a reflected radio-frequency or microwave signal that can reveal information about the environment. In preferred embodiments, the transmission system may be arranged for transmitting a microwave signal (e.g. a signal having a fundamental frequency in the range 300 MHz-300 GHZ), but may additionally or alternatively be able to transmit longer- or shorter-wavelength RF signals (e.g. a signal in the range 10 kHz-300 MHz). A parameter of the reflected RF or microwave signal is sensed and processed, allowing a property of the environment adjacent the knife blade to be determined. This arrangement can, at least in some embodiments, allow the presence of a particular material adjacent to the knife blade, such as tissue being cut, to be detected in real time. The system may enable properties of a material and/or of the progress of the cut to be determined. This feedback may be used to control an automated control system, e.g. comprising a robotic arm to which the knife blade is fastened, or to provide information to a human operator of the cutting system.

In some embodiments the knife blade comprises a first cutting edge, which may be straight or angled or curved. It may comprise one or more further cutting edges. It may comprise a second cutting edge, at least a portion of which may be parallel to at least a portion of the first cutting edge. The knife blade may comprise a sharpened tip, although this is not essential.

The knife blade may be partly formed of an electrically-conductive material, which may be metal, such as stainless steel. The electrically-conductive material may form a first conductive cutting edge of the knife blade.

The knife blade preferably provides a waveguide for guiding radio-frequency or microwave signals along the knife blade. The knife blade may comprise a first electrical conductor that provides a first cutting edge of the knife blade. It may comprise a second electrical conductor, which is preferably separated from the first electrical conductor by a first insulator region. The first and second electrical conductors and the first insulator region may form a first RF or microwave waveguide, which may provide part or all of a transmission line for the RF or microwave signal.

In a first set of embodiments, the second electrical conductor provides a second cutting edge of the knife blade. In such embodiments, the insulator region may be centred along an axis of the knife blade. It may define a tip of the knife blade.

In a second set of embodiments, the knife blade comprises a third electrical conductor, which preferably provides a second cutting edge of the knife blade. The third electrical conductor is preferably separated from the second electrical conductor by a second insulator region. The second and third electrical conductors and the second insulator region may form a second RF or microwave waveguide. In such embodiments, the second electrical conductor may be centred along an axis of the knife blade, and may define a tip of the knife blade. The first and third electrical conductors may be electrically coupled to each other. They may be coupled to a ground potential. The second electrical conductor may be coupled to a signal potential.

In both sets of the embodiments, the electrical conductors and the insulator region or regions may be elongate regions. They may extend along an axis of the knife blade. They may be parallel or substantially parallel to each other. They may be formed of respective substantially planar elements. They may be arranged to be coplanar, within a plane of the knife blade—i.e. arranged side-by-side across a width of the knife blade.

The knife blade, or at least an exposed portion thereof, may be less than 50 cm in length (i.e. along an axis of the blade). It may be between 1 cm and 30 cm in length, such as between 5 cm and 15 cm. However, the blade may have be longer or shorter than this in some embodiments, depending on cutting requirements.

The knife blade is preferably made only from non-toxic or food-safe materials.

The cutting system may comprise a knife holder, and the knife blade may be mounted to the knife holder. The knife holder and knife blade may form a knife assembly. The knife blade may be contained partly within a cavity of the knife holder. The knife holder may comprise coupling means, such as one or more apertures and/or bolts and/or fasteners, for coupling the knife holder to a mechanical actuator, such as a robotic arm. The knife holder may be formed from an electrical insulator, such as a polymer. In some preferred embodiments, the knife holder may be formed principally (e.g. at least 50% or 80% by mass) from polyether ether ketone (PEEK). This may advantageously provide a high degree of mechanical and chemical resistance, which may be beneficial in butchery or surgical applications.

The knife holder may comprise an impedance matching component, to which the knife blade and the RF or microwave transmission system are both electrically coupled. This component may comprise a printed circuit board (PCB), which may comprise a microstrip waveguide structure.

Some or all of the RF or microwave transmission system, the sensing system and the processing system may be external to the knife holder and knife blade.

The RF or microwave transmission system may comprise an RF or microwave signal generator, such as a microwave generator. The RF or microwave transmission system may comprise an RF or microwave transmission line, such as a coaxial cable, for communicatively coupling the RF or microwave signal generator and the knife blade. The RF or microwave transmission system may be arranged for transmitting an RF or microwave signal to the knife blade at a configurable one of a plurality of fundamental (i.e. carrier) frequencies. The RF or microwave signal may be an unmodulated signal. It may be a sine wave. It may have a fundamental frequency in the range 50 kHz-10 GHz, and preferably in the range 50 kHz-4.4 GHz. The RF or microwave transmission system may be arranged to transmit a sweep signal (e.g. a linear up-chirp or down-chip). The RF or microwave signal may sweep over a range of at least 10 MHz, or 100 MHz, or 1 GHz or more. However, in some embodiments, a narrow range may be sufficient and may advantageously allow for a faster update rate. Thus, in some embodiments the RF or microwave signal may sweep over a range of at most 100 MHz, or 10 MHz, or less. The RF or microwave signal frequency may be chosen at least partly based on a length of the knife blade.

The RF or microwave signal may have power less than 10 milliwatts, and preferably less than 1 mw.

The determined property of the environment may comprise one or more of: the presence of solid or liquid material adjacent (e.g. in contact with) the knife blade; a type of material adjacent the knife blade; and an extent of contact between material and the knife blade (e.g. a depth of cut).

The parameter may be any value derived from the reflected signal. It may be determined as one or more complex or scalar values. In some embodiments, the parameter may depend at least partly on the phase of the reflected signal. In some embodiments, the parameter may equal or be representative of the voltage standing wave ratio of the RF or microwave signal. The parameter may, in some preferred embodiments, equal or be representative of a scattering parameter (S-parameter) of the cutting system in the environment. It may equal or be representative of a reflection coefficient (e.g. an S₁₁ scattering parameter). The parameter may represent a ratio of the power or amplitude of the RF or microwave signal transmitted to the knife blade to the power or amplitude of the RF or microwave reflection signal. It may be determined as a complex value or as a scalar value. It may be represented or encoded as parameter data, which may have any appropriate format. It may be stored in a memory of the sensing system and/or of the processing system. It may be communicated between the sensing system to the processing system using any appropriate analog or digital signal or representation.

A value of the parameter may be determined for only a single frequency. However, in preferred embodiments, respective values of the parameter may be determined for a plurality of different RF or microwave frequencies. A plurality of such parameter values may be stored as frequency spectrum data in a memory of the processing system.

A plurality of values of the parameter may be determined over a measurement time interval and the frequency of the transmitted RF or microwave signal may be varied (e.g. stepped discretely or swept continuously) over the measurement time interval. The processing system may analyse the parameter for a plurality of different frequencies to determine the property of the environment.

The processing system may identify one or more peaks (which may be negative—i.e. dips) in the parameter over frequency. Such a peak may correspond to a resonant mode of the knife blade in the environment. The processing system may determine a frequency of a peak in the parameter. It may determine an amplitude of a peak in the parameter. It may use the determined frequency and/or amplitude when determining the property of the environment. It may perform vector network analysis or principle component analysis on the parameter data (e.g. representing values of the parameter determined at a plurality of different respective frequencies).

Although, in general, analysing the parameter across a wider range of frequencies may provide more information about the environment, in some embodiments the parameter may be evaluated only at a single frequency or a set of at most five, ten or a hundred frequencies. This may enable the property to be determined more quickly and/or with lower processing burden. This may allow the property to be determined repeatedly over time at a faster update rate. A transmission frequency or set of frequencies may be selected at which a relatively large change in a resonant frequency of the RF or microwave signal is expected, based on the type of environment in which the knife is expected to be used.

The processing system may store respective information representative of values of the parameter when the knife blade is in each of a plurality of different environments. The processing system may be configured, when determining the property of the environment, to compare determined parameter data (e.g. representing a respective value of the parameter at one or more respective frequencies) with one or more predetermined parameter templates, which may be stored in a memory of the processing system. Each parameter template may represent a different environment adjacent the knife blade—e.g. a different material type that the knife blade may be cutting. The processing system may determine a best matched template to the parameter data. It may compare an amplitude and/or frequency of a peak in the parameter data with the template data. It may perform a correlation operation. It may use machine learning, e.g. a trained neural network, to determine a best matched template.

The processing system may comprise a processor and a memory storing software for execution by the processor. The software may comprise instructions for analysing the parameter to determine said property.

The property of the environment may be determined repeatedly over time. It may be determined once for each measurement time interval of the RF or microwave transmission system. It may be determined at least every 500 ms or at least every 5 ms or more frequently, depending on requirements.

The processing system may output data to a mechanical control system (e.g. a robotic controller) for controlling a mechanical actuator for moving the knife blade. The cutting system may comprise a mechanical actuator for moving the knife blade in space (in one, two or three dimensions). It may comprise a robotic arm.

The processing system may additionally or alternatively output data relating to the property of the environment to a human user, who may be an operator of the cutting system (e.g. a butcher or a surgeon). The cutting system may comprise a display screen for displaying data representative of the determined property of the environment (e.g. for displaying a cutting depth, and/or for indicating one or more materials determined to be adjacent the knife blade).

In some embodiments, the method of operating a knife blade may comprise butchering an animal carcass. It may comprise determining the property of the environment adjacent the knife blade while using the knife blade to butcher an animal carcass.

In other embodiments, the method of operating a knife blade may comprise performing a surgical procedure on a human or animal patient. It may comprise determining the property of the environment adjacent the knife blade while using the knife blade to perform a surgical procedure on a human or animal patient.

Features of any aspect or embodiment described herein may, wherever appropriate, be applied to any other aspect or embodiment described herein. Where reference is made to different embodiments or sets of embodiments, it should be understood that these are not necessarily distinct but may overlap.

### BRIEF DESCRIPTION OF THE DRAWINGS

Certain preferred embodiments of the invention will now be described, by way of example only, with reference to the accompanying drawings, in which:

FIG. 1 is a schematic diagram of a cutting system embodying the invention, including a cutaway isometric perspective view of a knife of the cutting system;

FIG. 2 is an isometric perspective diagram showing the exterior of the knife;

FIG. 3 is a side view of the knife;

FIG. 4 is a vertical cross-sectional view along a length of the knife;

FIG. 5 is an exploded view of the knife;

FIG. 6 is an exemplary plot of reflected power against frequency, for a variety of tissue types, as measured using a cutting system embodying the invention; and

FIG. 7 is an exemplary plot of reflected power against frequency, for a variety of cutting depths, as measured using a cutting system embodying the invention.

### DETAILED DESCRIPTION

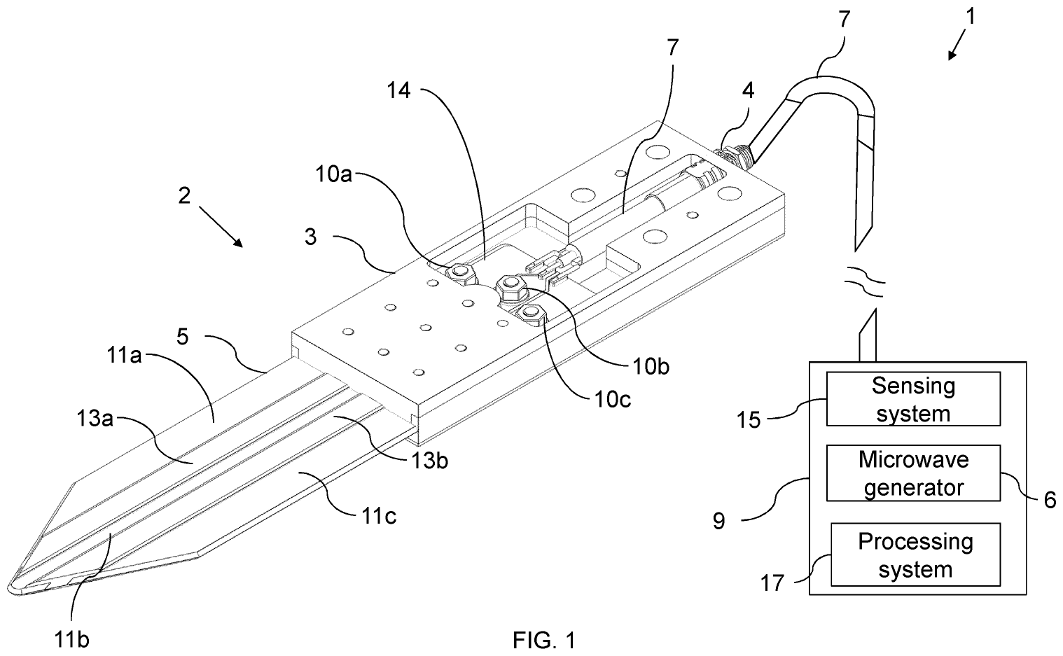

FIG. 1 is a schematic diagram of a cutting system 1 according to an embodiment of the present invention. The cutting system 1 comprises a knife assembly 2, comprising a knife holder 3 and a knife blade 5, which is coupled to a control system 9 by a microwave signal line 7. The microwave signal line 7 is used to transmit microwave signals (and optionally signals of other radio frequencies) to the knife assembly 2, and to receive reflected microwave (and RF) signals from the knife assembly 2.

Microwave signals are generated by a microwave generator 6 of the control system 9 and reflected microwaves are received by a sensing system 15 of the control system 9. The generation and sensing of microwave signals is controlled using a processing system 17 of the control system 9. In some embodiments, the sensing system 15 and the microwave generator 6 may be provided by a vector network analyser (VNA) device, and the processing system 17 may be provided by a laptop computer connected to the VNA. However, in other embodiments, the control system 9 may be implemented as a single device, or it may be distributed in any appropriate way.

The cutting system 1 utilises electromagnetic radiation to sense properties of material in the vicinity of the knife blade 5, which is employed as a sensing element. Microwave signals reflected along the knife blade 5 are analysed by the processing system 17 in order to determine properties of the environment in the vicinity of the knife blade 5 as will be described in the following. Employing the knife blade 5 as a sensing element during cutting allows information about the cutting process to be obtained from the knife blade 5 directly, without requiring the use of separate sensing components such as a computer vision system, which may be unable to obtain information from close to the surface of the knife blade 5, especially when the blade 5 is embedded in an optically-opaque medium such as animal tissue.

As shown in FIG. 1, the knife blade 5 comprises two outer elongate electrically-conductive sections 11a, 11c and a central elongate electrically-conductive section 11b. These are separated respectively by two elongate insulating sections 13a, 13b, formed of PEEK, arranged to provide electrical isolation between the three conductive sections 11a-11c. These sections 11a-11c, 13a-13b are arranged coaxially along an axis of the knife blade 5 and alternate across a plane of the knife blade 3. They are held together partly by mutual friction fit.

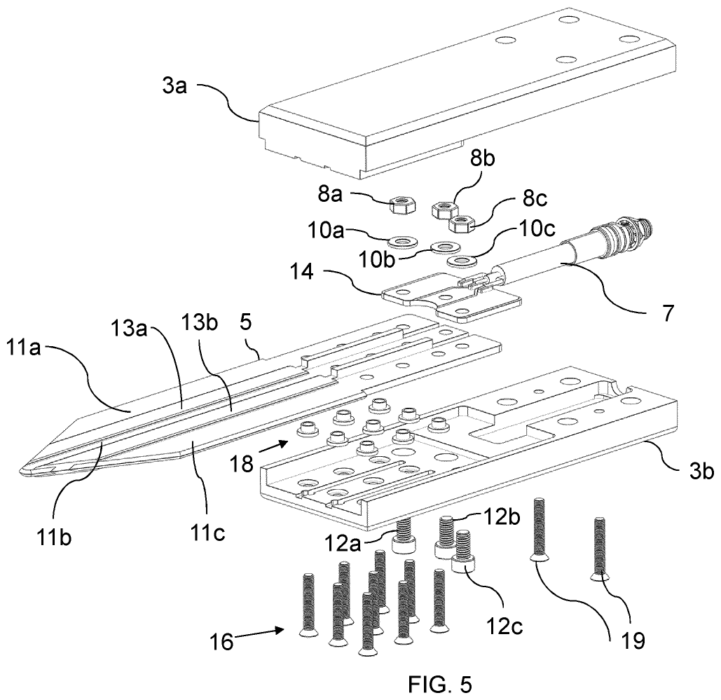

The three conductive sections 11a-11c are attached to the knife holder 3 by a set of nine screws 16, arranged in three lines of three screws. The sections 11a-11c are electrically isolated from the screws 16 by nine respective nylon spacers 18 (as shown in FIG. 5). The two outer conductive sections 11a, 11c at the edges of the knife blade 5 are sharpened such that they form respective cutting edges of the knife blade 5. The inner conductive section 11b defines the tip of the knife blade 5. The tip may be sharpened or blunt, depending on requirements.

The microwave signal line 7 is configured to transmit microwave signals between the knife blade 5 and the sensing system 15 and the microwave generator 6 of the control system 9. The term “microwave”, when used herein without further qualification, may be given a relatively broad interpretation. The microwave generator 6 is preferably capable of generating a signal having a fundamental frequency that can be selected to be anywhere in the range 150 kHz-10 GHz, although it may be able to generate RF or microwave signals having higher or lower wavelengths depending on requirements and the capabilities of the hardware employed in certain embodiments.

In the embodiment shown in FIG. 1, the microwave signal line 7 comprises a flexible coaxial cable, comprising inner and outer metal conductors separated by an insulating layer. The microwave signal line 7 enters the knife holder 3 through a strain-relief grommet 4 and terminates at a split out, which couples the microwave signal line 7 to a printed circuit board (PCB) 14 designed transmit microwave signals along the knife blade 5. The printed circuit board 14 is arranged with a coplanar microstrip waveguide structure for impedance matching, comprising three metalized sections on its upper face, each of which is in electrical contact with one of the three conductive sections 11a-11c of the knife blade 5, which are electrically isolated from each other by the dielectric substrate of the PCB 14. Each of the three conductive sections 11a-11c of the knife blade 5 is held in electrical contact with a respective metalized section of the PCB 14 by a respective nut 8a-8c, metal washer 10a-10c and metal bolt 12a-12c (shown in FIG. 5).

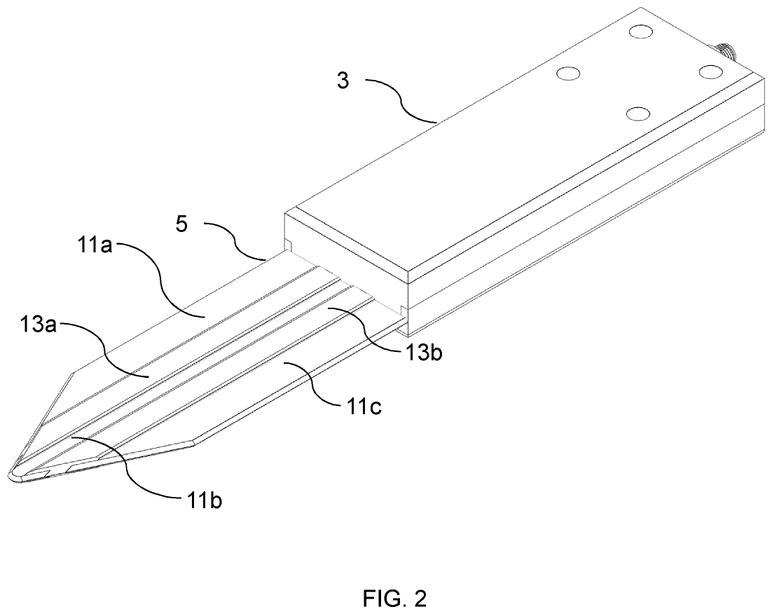

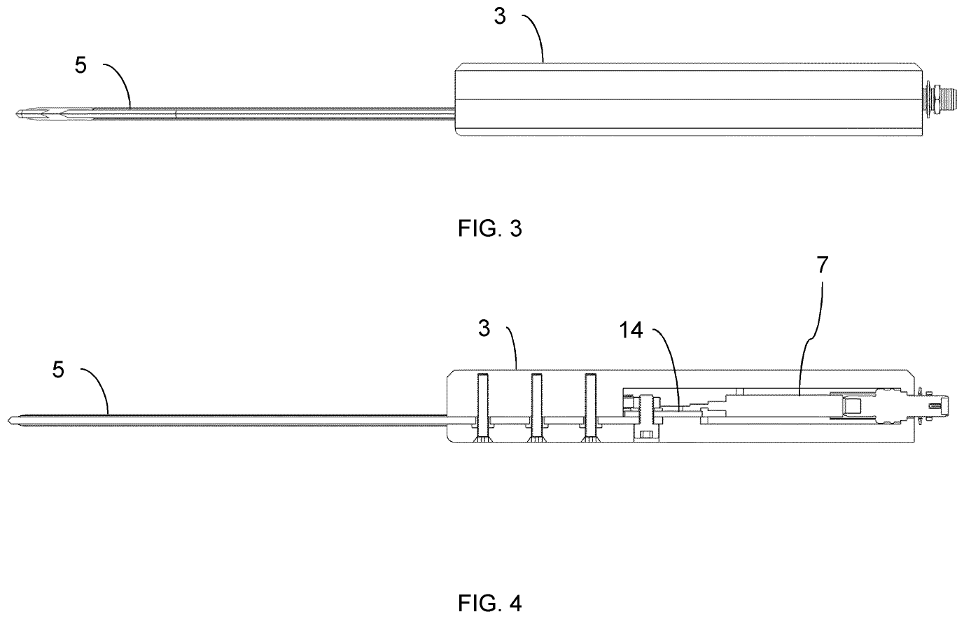

The relative positions of each of the components of the knife holder 3 and the knife blade 5 of the cutting system are illustrated in FIGS. 2 to 5.

The knife assembly 2 shown in FIGS. 1-5 is approximately 30 cm in length, with the knife holder 3 being approximately 15 cm long×5 cm wide×2 cm tall, and with the protruding portion of the knife blade 5 being approximate 15 cm long×5 cm wide (tapering at the tip)×2 mm thick (tapering at the sharpened edges). However it is anticipated that knife assemblies having smaller or greater dimensions of the knife blade 5 and/or knife holder 3 could be used without departing from the scope of the disclosure as defined by the appended claims. The knife holder 3 comprises an upper moulding 3a and a lower moulding 3b, both of PEEK, which are joined using a plurality of screws 19 (shown in FIG. 5). A proximal portion of the knife blade 5 and a terminal portion of the microwave signal line 7 are secured in a cavity within the knife holder 3 defined by the upper and lower mouldings 3a, 3b. When sealed, the knife holder 2 is arranged such that approximately 15 cm of the knife blade 5 protrudes from the knife holder 3 with approximately 8 cm being retained in the holder 2. The screws 19 that secure the knife holder 3 together may also allow the knife holder 3 to be mounted to external components. For example, the knife holder 3 can be attached to a robotic arm to enable the robotic arm to control the knife blade 3 of the cutting system 1. In such an implementation, data obtained from the control system 9 can be provided to a processor controlling the robotic arm in order to inform its operation.

The PCB 14 comprises pads for soldering the inner conductor of the microwave signal line 7 to the central conductive section 11b of the knife blade 5, and for soldering the outer conductor of the microwave signal line 7 to the two outer conductive sections 11a, 11c at the edges of the knife blade 5. In this way the outer conductive sections 11a, 11c at the edges of the knife blade 5 are held at a common potential difference relative to the inner conductive section 11b. In addition to providing this electrical coupling, the printed circuit board 14 may also serve for impedance matching, i.e. to provide a desired impedance to the microwave signal.

Microwave signals from the microwave signal line 7 are transmitted, via the soldered connections and PCB 14, to the knife blade 5. The insulating sections 13a, 13b of the knife blade 5 largely confine the electromagnetic field so as to form a pair of waveguides for the microwave signal within the knife blade 5 (i.e. acting as a transmission line for the microwave signals). The arrangement of the conductive sections 11a-11c and the insulating sections 13a, 13b functions in a manner comparable to a cross section of the coaxial microwave signal line 7, in that the outer conductive sections 11a, 11c are coupled and act as a first conductor held at ground, and the inner conductive section 11b acts as a central, second conductor. Microwave signals propagate along the knife blade 5 principally in the region between the conducting regions. However, a portion of the microwave signal emanates from the edges of the knife blade 5 and couples with the external environment in the vicinity of the knife blade 5.

Microwave signals transmitted from the knife blade 5 are absorbed or scattered by the environment in the vicinity of the knife blade 5 to varying degrees, depending on the properties of the environment and the wavelength of the microwave signal. Depending on the frequency of the transmitted microwave signal and the physical properties of the environment, more or less of the microwave radiation will be reflected back through the knife blade 5 and to the microwave signal line 7, which passes the reflected signal to the control system 9 for analysis.

The control system 9 comprises a sensing system 15 for sensing the scattered microwave signal, and a processing system 17 configured to analyse the sensed scattered signal. Analysis of the scattered signal can provide information about the environment adjacent to the knife blade 5, allowing it to be characterised as will be described in the following.

For a given frequency, if the power of the microwave signal transmitted to the knife blade 5 from the microwave generator 6 is known, the reflected signal amplitude may be used to determine a reflection-coefficient parameter for the system, representing the ratio of the complex or scalar amplitude or power of the reflected signal relative to the complex or scalar amplitude or power of the signal transmitted along the knife blade 5. As the amplitude of the reflected wave is dependent on the physical properties of the environment adjacent to the knife blade 5, it can be analysed by the control system 9 to determine properties of the surrounding environment.

By sampling the parameter repeatedly, analysis of the parameter can be performed in real-time, as the environment around the knife blade 5 changes, e.g. while material is being cut by the knife blade 5.

For example, if the knife blade 5 of the cutting system 1 is used to cut through animal tissue, the parameter will change as a cutting edge of the knife blade 5 passes through muscle, fat, or bone, due to the changing material adjacent the knife blade 5 (and potentially due to a changing depth of cut). The degree of change will vary with the frequency of the microwave signal, due to the different effects these materials will have on the resonant properties of the knife blade 5.

Based on the measured parameter, the processing system 17 may be able, in some embodiments, to detect when the knife blade 5 is in contact with a solid or semi-solid object, as opposed to air, and/or to determine information about the depth of the knife blade 5 within the object (when the blade is at least partially embedded in an object). The processing system 17 may also be able to determine information about the material composition of the object (e.g. whether the knife blade 5 is cutting through muscle, fat, or bone, or some combination of these).

If used in butchery, monitoring of the parameter can therefore help to prevent cutting into materials like bone, which is rarely desirable, and can have a negative impact on knife sharpness or durability.

It is also anticipated that the cutting system 1, or variants thereof, may be employed to perform surgery on humans or animals in some embodiments. In surgical applications the processing system 17 may monitor the parameter to determine differences between different tissue types as the knife blade 5 passes through tissue. For example, the cutting system 1 may be employed in the excision of unhealthy tissue, such as cancerous tissue. In such excisions, it can be important that all of the unhealthy tissue is removed while ensuring that healthy tissue is affected as little as possible. The cutting system 1 may be used to reduce the amount of healthy tissue, or tissue of types other than the type of the unhealthy tissue, that is damaged by monitoring the parameter during a surgical operation. During an excision, the processing system 17 may determine, based on the parameter, that the knife blade 5 is entering a region of healthy tissue, allowing the operator to arrest motion of the knife blade 5, preventing unnecessary damage to healthy tissue. Depth information may also be determined to better inform the operator of the position of the blade in the human or animal body, and the likelihood of penetration of the knife blade 5 into vulnerable areas.

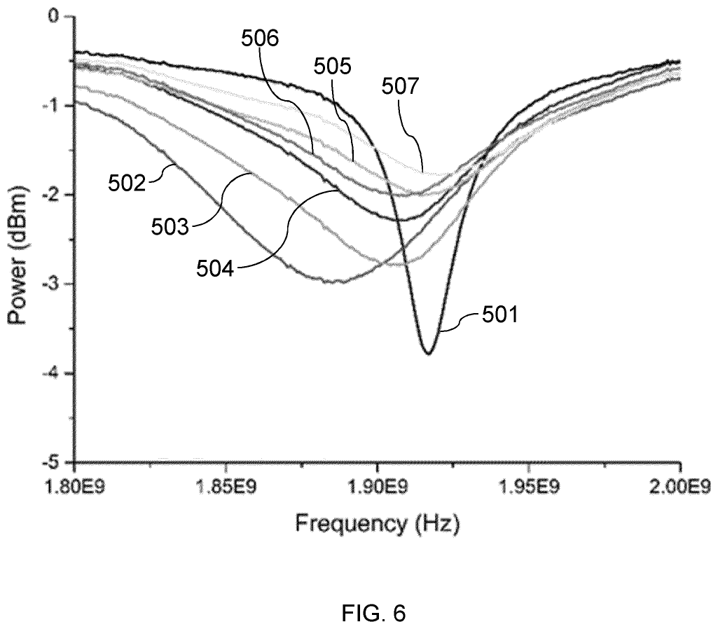

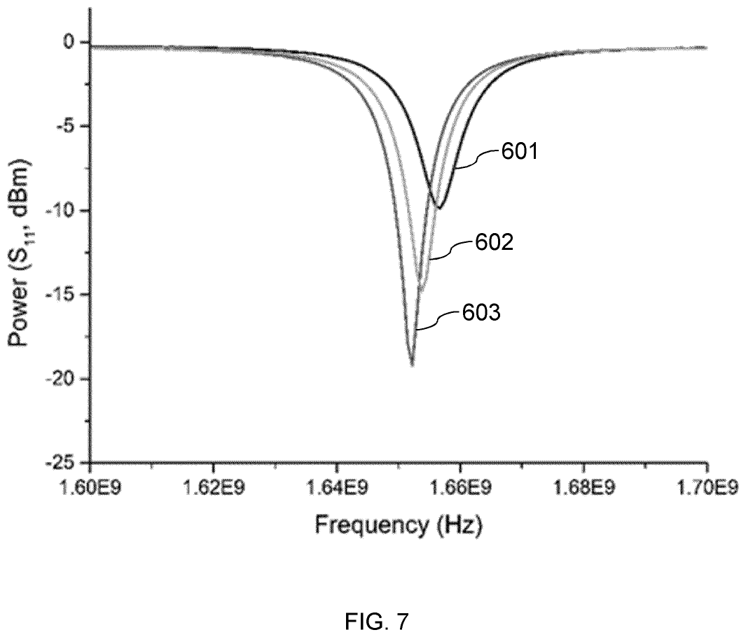

Measurement and analysis of the parameter by the processing system 17 will now be described with reference to FIGS. 6 and 7.

In use, at a first time t₀, an RF signal is generated by the microwave generator 6 at a first frequency f₀. This signal is transmitted through the microwave signal line 7 and the knife blade 5, and a portion of the signal is transmitted into the environment in the vicinity of the knife blade 5. A reflected (scattered) signal is received at the knife blade 5 from the environment, and is transmitted to the control system 9 through the microwave signal line 7. The sensing system 15 samples the reflected signal, which is converted to a digital signal and provided to the processing system 17. The processing system 17 compares the amplitude or power of the scattered signal to the known (predetermined) amplitude or power of the signal transmitted from the microwave generator 6 along the knife blade, to calculate a parameter value representative of a reflection coefficient (e.g. an S-parameter S₁₁) for the knife blade 5, for the frequency f₀ and time to. The value of this parameter, at the frequency f₀, is then stored in a memory of the processing system 17.

At a second time, t₀+Δt, a second microwave signal is generated by the microwave generator 6 at a second frequency f₀+Δf, and is transmitted through the signal line 7. The process described above is then repeated in order to evaluate the parameter at the second frequency f₀+Δf, and second time t₀+Δt. The parameter value at the frequency f₀+Δf is then also stored in the memory of the processing system 17. By sequentially generating microwave signals at a plurality of frequencies, and measuring the scattered signal at each frequency, a frequency spectrum of parameter values may be determined over a desired frequency range. The microwave generator 6 may be controlled to transmit each of a discrete plurality of different fundamental frequencies at different respective time periods within a sampling interval, or it may transmit a sweep (e.g. a continuous linear sweep) of frequencies over time, within a sampling interval. The processing system 17 may evaluate the parameter at one, two, ten, a hundred or a thousand or more frequencies over a frequency band (e.g. spaced uniformly across the interval 50 kHz to 4.4 GHZ, or at specific characteristic frequencies of unequal spacing).

The processing system 17 may compare the measured frequency spectrum to a set of predetermined spectra stored in a memory of the processing system 17 to determine one or more properties of the environment in the vicinity of the knife blade 5. The spectra stored in the memory may cover a range of material types and/or penetration depths, and may represent parameter values over one, two, ten, a hundred or a thousand or more frequencies. Comparison of the measured frequency spectrum with one or more spectra stored in a memory of the processing system 17 may be carried out using appropriate software executed by the processing system 17. Such software may be configured to determine a degree of similarity between the measured frequency spectrum and each of the spectra stored in a memory of the processing system 17. In this way, the stored spectrum most similar to the measured spectrum may be identified, and a determination can be made that the characteristics of the environment in the vicinity of the knife blade 5 are comparable to those of the environment in which the stored spectrum were recorded.

Software used by the processing system 17 for this purpose may employ any appropriate method to compare spectra. For example, the software may apply a correlation function to determine a correlation coefficient between the measured spectrum and each of the spectra stored in a memory of the processing system 17. The correlation coefficients may be ranked, and the stored spectrum corresponding to the highest correlation coefficient may be identified. The properties of the environment in the vicinity of the knife blade 5 may then be determined to be comparable to those of the environment in which the stored spectrum corresponding to the highest correlation coefficient were recorded. Software executed by the processing system 17 may apply pattern recognition techniques to identify the most similar spectra. The software may apply principal component analysis to the measured spectrum and the spectra stored in a memory of the processing system 17 to generate one or more principle components for each spectrum, which may be compared in place of entire spectra to allow the most similar spectra to be identified.

In some embodiments, the software may employ machine learning (such as a convolutional neural network) to perform spectrum matching in order to identify the most similar spectra.

Comparison of a measured frequency spectrum against stored spectra may allow determination of characteristics of the environment in the vicinity of the knife blade 5. For example, as described above, it may be possible to determine the presence of material adjacent the blade; the type of material adjacent the blade; and/or the depth of material along the blade.

The range of frequencies over which the parameter is measured to generate the frequency spectrum may be selected based on the expected environment in the vicinity of the knife blade 5. For example, when the cutting system 1 is used to cut through animal tissue, which predominantly comprises water, a frequency range may be selected to include one or more frequencies at which significant absorption of the microwave signal occurs, i.e. to include at least one resonant frequency of water molecules. At such a frequency, the scattered signal is reduced in proportion to the water content in environment in the vicinity of the knife blade 5 (i.e. an object being cut), producing a negative peak in the scattered signal spectrum. The selected frequency range may also be determined based on the length of the knife blade 5. If the length of the knife blade 5 is reduced (as may be beneficial when the cutting system 1 is employed in surgical applications) higher frequency signals are required as peaks in the scattered signal spectrum are shifted to higher frequencies. Similarly, if the length of the knife blade 5 is increased, as may be the case in certain butchery applications, lower frequency signals may be used, as peaks in the scattered signal spectrum are shifted to lower frequencies.

Selecting a frequency range that spans a known resonant peak may also allow a narrower frequency range to be sampled, while still allowing sufficient information about the environment in the vicinity of the knife blade 5 to be obtained. This can reduce the amount of processing required, and increase the speed at which frequency spectra can be recorded, both of which can facilitate a faster update rate. Rapid acquisition of frequency spectra allows real-time monitoring of the environment in the vicinity of the knife blade 5 with less lag and higher temporal resolution, which can be used to inform a human or robotic operator of the cutting system 1 more accurately about the cutting process. This may be particularly important when the cutting system 1 is paired with and controlled by a robotic arm, as rapid feedback on the cutting process can be provided to the robotic arm for more precise control of the knife blade 5. Achieving a high temporal resolution may also be important in the context of applying the cutting system 1 in surgical applications, such that any changes in tissue detected by the system are identified as soon as possible. For example, when excising tissue, it may be possible to immediately inform the operator of a boundary between different tissue types when the knife blade 5 crosses a tissue boundary, preventing unnecessary excision of healthy tissue.

FIG. 6 shows an example of seven frequency spectra recorded by the cutting system 1, plotted as the (scalar) magnitude of the complex logarithm reflection coefficient, S₁₁, against frequency, in which the knife blade 5 is inserted into water to seven different depths along the knife blade 5. Curve 501 shows a frequency spectrum measured by the cutting system 1 while the knife blade 5 is held entirely in air, in which a peak can be observed at ˜1.92 GHz. Curve 502 shows a frequency spectrum measured with just the tip of the knife blade 5 in contact with water. Curves 503-507 show the measured frequency spectrum recorded with the knife blade 5 inserted into water to 20 mm, 40 mm, 60 mm, 80 mm and 100 mm respectively.

It can be seen from the curves 501-507 that significant changes in the frequency spectrum are observed, both in peak amplitude and the frequency at which the peak is observed.

It is clear from FIG. 6 that significant changes in the measured spectra occur with changes in the insertion depth of the knife blade 5. Storing spectra representative of different knife blade insertion depths (and potentially for each of a plurality of different materials) in a memory of the processing system 17 and comparing measured spectra to the stored spectra can therefore allow determination of insertion depth based on comparison of measured parameter values with those stored in a memory.

By comparing measured peaks to the same peaks in the spectra stored in a memory of the processing system 17, characteristics of the environment in the vicinity of the knife blade 5 may therefore be determined, e.g. based on knowledge of the relative water content in the materials being cut. In the context of butchery, as the relative water contents of different tissue types are well-known, such a comparison may allow the presence of muscle, fat, or bone to be determined in the vicinity of the knife blade 5.

Although the example of water is provided above, it is also possible to determine material properties based on other peaks in the scattering spectra which vary with changing material properties. An example of this is shown in FIG. 7, which shows a scattering peak at ˜1.65 GHz recorded with the knife blade 5 in contact with an animal carcass. Curve 601 shows a frequency spectrum measured by the cutting system 1 while the knife blade 5 is held entirely in air, not in contact with any object. Curve 602 shows a frequency spectrum measured by the cutting system 1 while the knife blade 5 is placed in contact with skin tissue of the animal carcass. Curve 603 shows a frequency spectrum measured by the cutting system 1 while the knife blade 5 is placed in contact with muscle tissue of the animal carcass. It can be clearly seen that the peak shifts in both amplitude and frequency depending on the material in the vicinity of the knife blade 5.

The frequency and amplitude of the peak shown in FIG. 7 may therefore be used to characterize the environment in the vicinity of the knife blade 5. For example, a measured spectrum may be compared with the spectra shown in FIG. 7 measured with the knife blade 5 in contact with fat or muscle tissue, and a determination may be made that measured spectra indicates that the knife blade 5 is in contact with fat and/or muscle tissue.

In some embodiments, two or more different parameters of the reflected radio-frequency signal may be sensed and used to determine information about the environment around the knife blade—e.g. two different types of scattering parameter.

In a variant set of embodiments, the knife blade may have only two conductive sections 11a, 11b, separated by a single insulating section 13a—i.e. not having a third conductive section 11c (electrically coupled to the first section 11a), nor a second insulating section 13b. In such arrangements, the knife could have only a single cutting edge, defined by the first conductive section 11a, or the second conductive section 11b could also be sharpened to form a second cutting edge.

It will be appreciated by those skilled in the art that the invention has been illustrated by describing one or more specific embodiments thereof, but is not limited to these embodiments; many variations and modifications are possible, within the scope of the accompanying claims.

1. A cutting system comprising:
a knife blade;
a radio-frequency or microwave transmission system for transmitting a radio-frequency or microwave signal to the knife blade;
a sensing system for sensing a parameter of a reflection of the radio-frequency or microwave signal; and
a processing system configured to analyse the parameter to determine a property of an environment adjacent the knife blade.

2. The cutting system of claim 1 wherein the knife blade provides a waveguide for guiding radio-frequency or microwave signals along the knife blade.

3. The cutting system of claim 1, wherein the knife blade comprises a first electrical conductor and a second electrical conductor, wherein the first electrical conductor provides a first cutting edge of the knife blade, wherein the second electrical conductor is separated from the first electrical conductor by a first insulator region, and wherein the first electrical conductor, the second electrical conductor, and the first insulator region form a first radio-frequency or microwave waveguide.

4. The cutting system of claim 3, wherein the first electrical conductor, the second electrical conductor, and the first insulator region are elongate and each extend along an axis of the knife blade.

5. The cutting system of claim 3, wherein the first electrical conductor, the second electrical conductor, and the first insulator region are formed of respective substantially planar elements and arranged to be coplanar within a plane of the knife blade.

6. The cutting system of claim 3, wherein the second electrical conductor provides a second cutting edge of the knife blade and wherein the first insulator region is centred along an axis of the knife blade.

7. The cutting system of any claim 3, wherein the knife blade comprises a third electrical conductor, separated from the second electrical conductor by a second insulator region, wherein the second electrical conductor, the third electrical conductor, and the second insulator region form a second radio-frequency or microwave waveguide, and wherein the third electrical conductor provides a second cutting edge of the knife blade.

8.-9. (canceled)

10. The cutting system of claim 7, wherein the first, second and third electrical conductors, and the first and second insulator regions, are elongate and each extend along an axis of the knife blade.

11. The cutting system of claim 7, wherein the second electrical conductor is centred along an axis of the knife blade.

12.-16. (canceled)

17. The cutting system of claim 1, wherein the radio-frequency or microwave transmission system is configured to transmit a sweep signal over a range of frequencies, and wherein the processing system is configured to analyse the parameter for a plurality of different frequencies when determining the property of the environment.

18. The cutting system of claim 1, wherein the processing system is configured to compare the parameter to a set of one or more predetermined parameter templates stored in a memory of the processing system when determining the property of the environment.

19. The cutting system of claim 1, wherein the processing system is configured to identify one or more peaks in the parameter over frequency when determining the property of the environment.

20. The cutting system of claim 19, wherein the processing system is configured to determine a frequency or an amplitude of the identified one or more peaks and to determine the property of the environment at least partly in dependence on the frequency or amplitude of the identified one or more peaks.

21. The cutting system of claim 19, wherein the processing system is configured to perform principle component analysis on parameter data representative of values of the parameter determined at a plurality of different respective frequencies.

22. The cutting system of claim 1, further comprising a mechanical actuator for moving the knife blade in space, wherein the processing system is configured to output data to a mechanical control system for controlling the mechanical actuator.

23.-25. (canceled)

26. The cutting system of claim 1, wherein the determined property of the environment adjacent the knife blade is an extent of contact between the knife blade and material adjacent the knife blade.

27. The cutting system of claim 1, wherein the determined property of the environment adjacent the knife blade is the presence of material adjacent the knife blade.

28. The cutting system of claim 1, wherein the determined property of the environment adjacent the knife blade is a type of material adjacent the knife blade.

29. A method of operating a knife blade, comprising:
transmitting a radio-frequency or microwave signal to the knife blade;
sensing a parameter of a reflection of the radio-frequency or microwave signal; and
analyzing the parameter to determine a property of an environment adjacent the knife blade.

30. The method of claim 29, comprising determining the property of the environment adjacent the knife blade while using the knife blade to butcher an animal carcass.
